(19) United States
(12) Patent Application Publication
(10) Pub. No.: US 2020/0376211 A1
(43) Pub. Date: Dec. 3, 2020

(54) AEROSOL PROVISION DEVICE AND APPARATUS FOR A VESSEL

(71) Applicant: BRITISH AMERICAN TOBACCO (INVESTMENTS) LIMITED, London (GB)

(72) Inventors: Alfred SPENCER, London (GB); Kevin BLICK, London (GB); Connor BRUTON, London (GB); Anna AZZOPARDI, London (GB); Julie Jenson BENNETT, London (GB); Kelly REES, London (GB)

(21) Appl. No.: 16/612,145

(22) PCT Filed: May 9, 2018
(86) PCT No.: PCT/EP2018/062101

§ 371 Date: Nov. 8, 2019

(30) Foreign Application Priority Data
May 9, 2017 (GB) 1707436.0

(51) Int. Cl. A61M 15/06; H05B 1/02

(52) U.S. Cl. CPC A61M 15/06; A61M 15/0021; H05B 1/0244

(57) ABSTRACT

An aerosol provision device for generating an inhalable aerosol comprises a housing containing a first chamber for containing a hot liquid. The housing also contains a second chamber for containing a substance that is heatable to generate the aerosol. The housing also contains a thermal energy transfer component for contacting the hot liquid when the hot liquid is contained in the first chamber to transfer thermal energy from the hot liquid to the substance when the substance is contained in the second chamber in order to heat the substance to generate the aerosol. The device comprises a mouthpiece comprising an outlet through which the aerosol may flow.

### PRIORITY CLAIM

The present application is a National Phase entry of PCT Application No. PCT/EP2018/062101, filed May 9, 2018, which claims priority from GB Patent Application No. 1707436.0, filed May 9, 2017, each which is hereby fully incorporated herein by reference.

### TECHNICAL FIELD

The present disclosure relates to an aerosol provision device and an apparatus for a vessel.

### BACKGROUND

Smoking articles such as cigarettes, cigars and the like burn tobacco during use to create tobacco smoke. Attempts have been made to provide alternatives to these articles that burn tobacco by creating products that release compounds without burning.

Examples of such articles are heating devices which release compounds by heating, but not burning, the material. The material may be for example tobacco or other non-tobacco products, which may or may not contain nicotine.

Apparatus is therefore known that heats tobacco or non-tobacco products to volatilize at least one component of the tobacco or non-tobacco products, typically to form an aerosol which can be inhaled, without burning or combusting the tobacco or non-tobacco products. Such apparatus is sometimes described as a “heat-not-burn” apparatus or a “tobacco heating product” (THP) or “tobacco heating device” or similar. Various different arrangements for volatilizing at least one component of tobacco or non-tobacco products are known.

Known THP devices have batteries for powering the heater of the THP device to volatilize tobacco contained within said device. Known THP devices also comprise electronics for controlling the heater in the device. This can lead to the requirement of large battery compartments containing a battery and electronics for powering the device, alongside the need to regularly recharge devices or supply new batteries.

### SUMMARY

According to a first aspect of the present disclosure, there is provided an aerosol provision device for generating an inhalable aerosol, the aerosol provision device comprising: a housing containing: a first chamber for containing a hot liquid; a second chamber for containing a substance that is heatable to generate the aerosol; and a thermal energy transfer component for contacting the hot liquid when the hot liquid is contained in the first chamber to transfer thermal energy from the hot liquid to the substance when the substance is contained in the second chamber in order to heat the substance to generate the aerosol; a mouthpiece comprising an outlet through which the aerosol may flow.

In an embodiment, the mouthpiece is connected to the housing.

In an embodiment, the arrangement is such that in use the housing is closed to prevent a user from accessing the first chamber.

In an embodiment, the first chamber comprises: an opening to provide access to the first chamber; and, a closing mechanism for engaging with the opening, the arrangement being such that the closing mechanism engages with the opening to prevent access to the first chamber through the opening.

In an embodiment, the thermal energy transfer component prevents liquid held in use in the first chamber passing into the second chamber.

In an embodiment, the second chamber is arranged towards a proximal end of the thermal energy transfer component.

In an embodiment, the second chamber is formed integrally with the thermal energy transfer component.

In an embodiment, the thermal energy transfer component is a heat pipe.

In an embodiment, the substance that is heatable to generate an aerosol comprises tobacco.

In an embodiment, the outlet of the mouthpiece is an adjustable outlet for controlling the amount of aerosol flowing through the outlet.

In an embodiment, the device is a hot beverage vessel and in use the first chamber contains a hot beverage.

In an embodiment, the aerosol provision device comprises an opening in fluid communication with the vessel, the arrangement being such that in use liquid held in the vessel may pass through the opening.

In an embodiment, the opening has a valve, the arrangement being such that in use liquid held in the vessel may pass through the valve.

In an embodiment, the housing comprises at least one vent, the arrangement being such that air may pass through the vent into the second chamber from outside the apparatus and from the second chamber to the mouthpiece.

According to a second aspect of the present disclosure, there is provided apparatus for a vessel for containing a hot liquid, the apparatus comprising: a lid for the vessel; a chamber for containing a substance that is heatable to generate an aerosol; a thermal energy transfer device for transferring thermal energy from the hot liquid when the hot liquid is contained in the vessel to the substance when the substance is in the chamber to heat the substance to generate the aerosol; and an outlet for the lid through which aerosol generated by the substance when heated can flow.

In an embodiment, the lid comprises an opening the arrangement being such that in use the vessel is in fluid communication with the opening.

In an embodiment, the opening has a valve, the arrangement being such that in use liquid held in the vessel may pass through the valve.

In an embodiment, the lid comprises at least one vent, the arrangement being such that air may pass through the vent into the apparatus from outside the apparatus.

In an embodiment, the outlet is an adjustable outlet for controlling the amount of aerosol flowing through the outlet.

In an embodiment, the vessel is a drinking vessel.

In an embodiment, in use the vessel contains a hot beverage.

In an embodiment, the thermal energy transfer device is a heat pipe.

In an embodiment, the substance that is heatable to generate an aerosol comprises tobacco.

In an embodiment, the thermal energy transfer device is releasably connectable to the lid.

In an embodiment, the chamber is arranged towards a proximal end of the thermal energy transfer device.

According to a third aspect of the present disclosure, there is provided a cartridge for use with an aerosol provision device for generating an inhalable aerosol; the cartridge comprising: a first chamber for containing a substance that is heatable to generate the aerosol, a thermal energy transfer component which, in use, is between the first chamber and a second chamber containing a hot liquid, the thermal energy transfer component for transferring thermal energy from hot liquid in the second chamber to the substance in the first chamber.

In an embodiment, the cartridge comprises the second chamber.

In an embodiment, the thermal energy transfer device is a wall of the first chamber or is a heat pipe.

In an embodiment, the second chamber is part of the aerosol provision device that the cartridge is inserted into in use.

According to a fourth aspect of the disclosure, there is provided a system for generating an inhalable aerosol, the system comprising: the cartridge of the third aspect; and an aerosol provision device into which the cartridge is insertable.

Further features and advantages of the disclosure will become apparent from the following description of preferred embodiments of the invention, given by way of example only, which is made with reference to the accompanying drawings.

### BRIEF DESCRIPTION OF THE DRAWINGS

FIG. 1 shows a schematic cross section of an aerosol provision device according to an example.

FIG. 2 shows a schematic cross section of an aerosol provision device according to an example.

FIG. 3A shows a schematic cross section of an aerosol provision device according to an example.

FIG. 3B shows a schematic side view of a portion of an aerosol provision device according to an example.

FIG. 3C shows a schematic end view of a portion of an aerosol provision device according to an example.

FIG. 4 shows a schematic cross section of an aerosol provision device according to an example.

FIG. 5 shows a cross section of an aerosol provision device according to an example.

### DETAILED DESCRIPTION

As used herein, the term “substance to be heated to generate an aerosol”, or similar terms, includes materials that provide volatilized components upon heating, typically in the form of an aerosol. The substance may include any tobacco-containing material and may, for example, include one or more of tobacco, tobacco derivatives, expanded tobacco, reconstituted tobacco or tobacco substitutes. Such a substance may also include other, non-tobacco, products, which, depending on the product, may or may not contain nicotine. The substance may for example be in the form of a solid, a liquid, a gel or a wax or the like. The substance may for example also be a combination or a blend of materials.

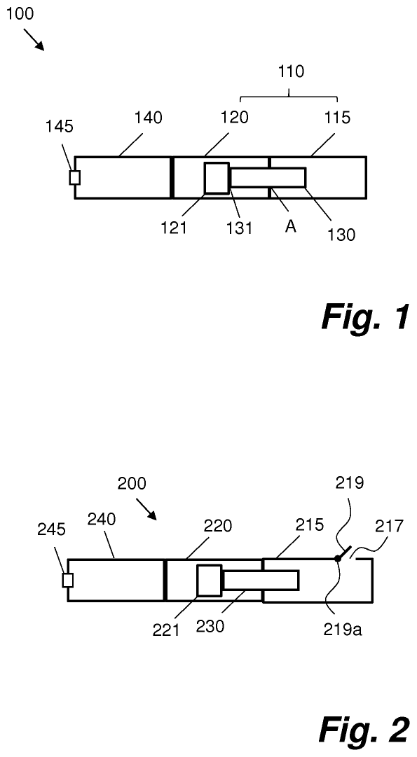

Referring to FIG. 1 there is shown a schematic cross section of an example of an aerosol provision device 100. The aerosol provision device 100 is an inhalation device 100 (i.e. a user uses it to inhale an aerosol provided by the device). The aerosol provision device 100 is hand-held. The device 100 has a housing 110 which contains a first chamber 115 for containing a hot liquid and a second chamber 120 for containing a substance 121 that is heatable to generate an aerosol and/or vapor. The device 100, in the example shown in FIG. 1, also has a thermal energy transfer component 130 for contacting the hot liquid when the hot liquid is contained in the first chamber 115 to transfer thermal energy from the hot liquid when the substance 121 is in the second chamber 120 in order to heat the substance 121 to generate the aerosol. In the example shown, the device 100 also has a mouthpiece 140 which has an outlet 145 through which the aerosol and/or vapor may flow.

In this respect, first it may be noted that, in general, a vapor is a substance in the gas phase at a temperature lower than its critical temperature, which means that for example the vapor our can be condensed to a liquid by increasing its pressure without reducing the temperature. On the other hand, in general, an aerosol is a colloid of fine solid particles or liquid droplets, in air or another gas. A colloid is a substance in which microscopically dispersed insoluble particles are suspended throughout another substance.

For reasons of convenience, as used herein the term aerosol should be taken as meaning an aerosol, a vapor or a combination of an aerosol and vapor.

In the example of the device 100 shown in FIG. 1, the device 100 in use has hot liquid contained in the first chamber 115. The heat from the liquid in the first chamber 115 may be conducted to the substance 121 that is heatable to generate an aerosol. The thermal energy transfer component 130 conducts heat from the hot liquid in the first chamber 115 to the substance 121 in the second chamber 120. The thermal energy component 130 may take any suitable form, for example, any of a heat pipe, a piece of thermally conducting material which may or may not be solid. In an example, the heat pipe may be made of metal or metals. In a specific example, the heat pipe may be a tin plated copper heat pipe. The heat pipe internals may use a sintered core to wick the working fluid, held in the heat pipe, towards the part of the heat pipe that is located within the first chamber 115. In an example, the thermal energy transfer component 130 may be a thermally conductive member such as a wall arranged between the first chamber 115 and the second chamber 120. The wall may be made of for example metal or metals.

In some preferred examples, the thermal energy component 130 is a heat pipe. As is known, a typical heat pipe comprises a sealed pipe partially filled with a working fluid. When one end or section of the heat pipe is heated (for example by being immersed in a hot liquid) the working fluid inside the heat pipe at the heated end or section evaporates creating a vapor pressure difference between that end or section of the heat pipe and an opposite cooler end or section of the heat pipe. This pressure difference causes a transfer of vapor from the heated end or section to the cooler end or section where the vapor condenses on an internal surface of the heat pipe releasing its latent heat and hence heating the cooler end or section of the heat pipe. The condensed working fluid then flows back to the heated end or section of the heat pipe. Due to high heat transfer coefficients for boiling and condensation, heat pipes are highly effective thermal conductors. In examples described herein, a suitable working fluid is water. Water allows for temperatures of up to 100 degrees to be achieved. The working fluid should be selected to be able to create enough heat to heat a substance 121 held in the second chamber 120 to generate an aerosol.

The thermal energy component 130 in a specific example prevents liquid held in use in the first chamber 115 passing into the second chamber 120. The thermal energy component 130 may fit snuggly in an opening between the first chamber 115 and the second chamber 120 such that no liquid can move from the first chamber 115 to the second chamber 120. If the liquid in the first chamber 115 were to contact the substance 121 contained in use in the second chamber 120 the heat capacity of the combined substance 121 and liquid may adversely affect the generation of aerosol from the substance 121. This is prevented in the arrangement shown in FIG. 1.

In the example shown in FIG. 1, the mouthpiece 140 is connected to and is part of the housing 110. In this example, the first chamber 115, the second chamber 120 and the mouthpiece 140 are arranged such that liquid held in the first chamber 115 in use is prevented from exiting the device 100. In an example, the mouthpiece 140 is suitable for being received in a mouth of the user to allow the user to draw on the mouthpiece 140. In an example, the mouthpiece 140 is integral with the housing 110 and may be formed from the proximal end of the housing 110.

In the example shown in FIG. 1, the arrangement is such that, in use, the housing 110 is closed to prevent a user from accessing the first chamber 115. This arrangement can improve the overall safety of the device 100. The device 100 disclosed herein may be used by a user during transit. Use of the device 100 during transit, or during any movement, increases the risk of spilling the hot liquid from the device 100. Liquid may be prevented from exiting the first chamber 115 when the housing 110 is closed and this, in turn, improves the safety of the device 100. In the example shown in FIG. 1, there exists no route out of the device 100 for hot liquid contained in the first chamber 115. The hot liquid may not pass through the thermal energy transfer component 130. The hot liquid may also not pass at the join A between the thermal energy transfer component 130 and the mouthpiece 140 as the thermal energy transfer component 130 fits snuggly into the opening between the first chamber 115 and the second chamber 120 as described above. Therefore, the hot liquid is safely contained within the first chamber 115 and the user is not at a risk of contacting the hot liquid. This arrangement also enables use “on the go”.

In some examples, prior to use, a user of the device 100 loads substance 121 into the second chamber 120 and fills the first chamber 115 with hot liquid, e.g. hot water. Heat from the hot water is conducted by the thermal energy transfer component 130 to the substance 121, generating an aerosol from the substance 121. The user then draws on the mouthpiece 140 and aerosol flows out the outlet 145 for inhalation by the user. The device 100 has air inlets (not shown) arranged such that airflow from external to the device 100, through the second chamber 120, mouthpiece 140 and outlet 145 can be achieved by inhaling on the mouthpiece 140.

In an example, the substance 121 may be contained in a pod which the user can load into the second chamber 120. In an example, the user may load the substance 121 directly into the second chamber 120 in the form of loose material, e.g. shag or fine strands of substance 121. In a specific example, the second chamber 120 is a volume of space for receiving the substance 121. In a specific example, the second chamber 120 is defined by a proximal end 131 of the thermal energy transfer component 130.

In an example wherein the second chamber 120 is defined by the proximal end 131 of the thermal energy component 130, the proximal end 131 may be arranged to receive a pod or loose substance 121. The proximal end 131 of the thermal energy component 130 may define a container with a closable lid such that the pod or loose substance can be inserted into the container and the lid closed to prevent the substance 121 exiting the second chamber 120. The lid or body of the container may have small perforations to allow aerosol to exit the container during use.

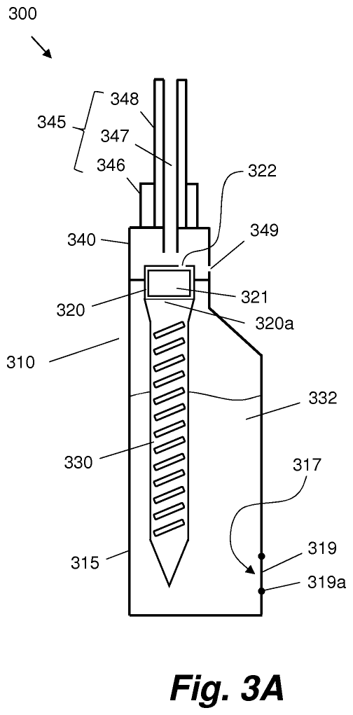

In the example shown in FIG. 2, the device 200 has a first chamber 215 which comprises an opening 217. The opening 217 provides access to, and an exit from, the first chamber 215. In an example, the opening 217 enables a user to access the first chamber 215. The user can refill the liquid in the first chamber 215 by, first, removing any cool liquid from the first chamber 215 and, second, refilling the first chamber 215 with hot liquid. Removal of the liquid from the first chamber 215 may be achieved by tipping the device 200 such that the liquid drains out of the opening 217. The opening 217 may have a funnel, channel, chute, slide, trough or similar for guiding hot liquid into the first chamber 215. This arrangement enables easy refilling of the first chamber 215 by the user. This also reduces the risk of hot liquid contacting the user when the user fills the device with hot liquid.

In the example shown in FIG. 2, the first chamber 215 has a closing mechanism 219 for engaging with the opening 217. The arrangement of the opening 217 and the closing mechanism 219 is such that the closing mechanism 219 may engage with the opening 217 to prevent access to the first chamber 215 through the opening 217. The closing mechanism 219 may be operated by the user to temporarily provide access to, and an exit from, the first chamber 215. In an example, the closing mechanism 219 is a door which can be operated by a user to block the opening 217. The door may be operated by a rotational motion around an axis or hinge, for example hinge 219a in FIG. 2. The door may be operated in a sliding motion across a side of the chamber 215 to block the opening 217. In another example the opening 217 is a hole in the first chamber 215 and the closing mechanism 219 is a plug of a size to fit snuggly in the hole and prevent passing of liquid into or out of the first chamber 215 when the plug is engaged with the opening 217. During use, the closing mechanism 219 may be engaged with the opening 217 to prevent liquid from passing out of the first chamber 215.

The closing mechanism 219 may additionally or alternatively be operated by the user pulling the closing mechanism 219 from the opening 217, or depressing the closing mechanism 219 in such a manner that the closing mechanism is then ejected out of the opening 217 by, for example, a biasing member arranged within the first chamber 215, or sliding the mechanism 219 laterally into and out of position to block the opening 217, or by operating an electronic system which performs any of the above.

Referring still to FIG. 2, the second chamber 220 is arranged towards the proximal end of the thermal energy transfer component 230. The second chamber 220, in use, contains the substance that is heatable to generate an aerosol. The second chamber 220 may be arranged towards the proximal end of the thermal energy transfer component 230, so as to be closer to the outlet 245 of the device 200. Aerosol generated from the substance will cool during transit through the device 200. The aerosol will, therefore, lose less heat by traversing a shorter distance to the outlet 245 of the device 200. The second chamber 220 may be arranged within the device 200 to ensure the aerosol arrives at the outlet 245 at a temperature suitable for inhalation by a user. In an example, the second chamber 220 is an integral part of the thermal energy transfer component 230. In an example, the second chamber 220 is not an integral part of the thermal energy transfer component 230 but is arranged at the end of the thermal energy transfer component 230 as shown in FIG. 2.

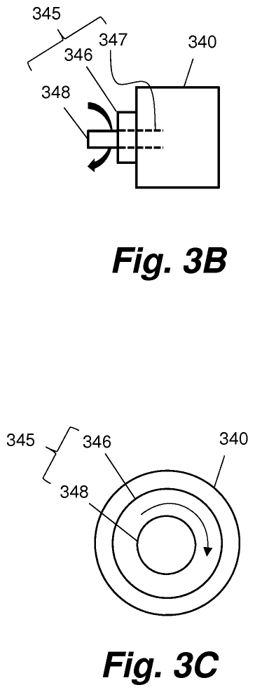

Referring now to FIG. 3A, there is shown a schematic drawing of an aerosol provision device 300. Features of the device 300 which are similar to features of previous examples, have been labeled with similar numerals with the numerals starting at 300. Description regarding these similar features may not be repeated here for concision.

The device 300 comprises a housing 310 that contains a first chamber 315 for holding a hot liquid 332, for example hot water. Hot liquid 332 can be inserted into the device through opening 317, which can be accessed via closing mechanism 319. The closing mechanism 319 and hinge 319a provide a liquid tight closing when closed, to prevent liquid exiting the first chamber 315.

The device 300 has a thermal energy transfer component 330, which in this example is a heat pipe, having a volume of space defined by a proximal end of the heat pipe 330 which acts as the second chamber 320. Substance 321 is held in second chamber 320 and the second chamber 320 is sealed off by an internal wall 320a from the section of the heat pipe 330 that contains its working fluid. The substance 321 may be contained in a flavor pod or container that is placed into the second chamber 320 or in the form of loose material which may comprise tobacco that is placed into the second chamber 320. If the substance 321 is contained within a flavor pod, the flavor pod is provided with a pod outlet (not shown) to enable aerosol to flow out of the flavor pod. The pod outlet (not shown) may be created by the user prior to inserting the flavor pod into the second chamber 320 (for example, by piercing a foil lid of the pod) or the device 300 may be provided with some means (not shown) for creating the pod outlet (shown) when the flavor pod is inserted into the second chamber 320.

The second chamber 320 has a fluid outlet 322 which allows aerosol generated in the second chamber 320 to exit the second chamber 320. The fluid outlet 322 may be opened and closed as desired by the user. In an example, the fluid outlet 322 is opened by the user drawing on the mouthpiece 340.

The mouthpiece 340 and outlet 345 are arranged at the proximal end of the device 300. The outlet 345 has a body portion 346, a passage 347 and an end portion 348. The mouthpiece 340 has an air inlet 349 to allow air into the device 300. The second chamber 320, the mouthpiece 340 and the passage 347 are in fluid communication. This fluid communication enables aerosol generated in the second chamber 320 to be mixed with air entering the device 300 through air inlet 349 prior to inhalation by the user.

The device 300 is a so called tobacco heating product (THP) as the substance 321 is heated to produce aerosol but is not burned during this process. The device 300 may have the external appearance of a typical THP device but whereas a typical THP device has a battery compartment containing a battery and electronics for operating the device, the battery chamber has been replaced with a chamber for containing a hot liquid. This removes a requirement for the device to be charged or have batteries replaced.

The example of a device 300 shown in FIG. 3A, may be modular. Each of the elements of the device 300 may be removed from the device 300 and changed. This enables a user to increase the overall lifetime of the device 300 by selectively replacing those elements that need replacing first, rather than buying a new device 300.

Referring now to FIGS. 3B and 3C, there are shown schematic drawings of an outlet 345 of an aerosol provision device 300. In the example shown, the outlet 345 is an adjustable outlet 345. Heat energy transferred from the beverage heats the substance to generate an aerosol that the user can then inhale through an outlet of a lid of the vessel. The adjustable outlet 345 can be adjusted by the user to adjust the size of the outlet. The adjustable outlet 345 can therefore control the flow rate of aerosol from the device to the user.

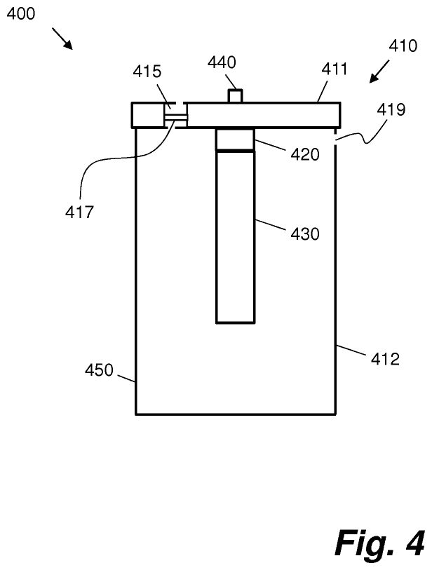

Referring now to FIG. 4, there is shown a schematic cross section of another example of an aerosol provision device 400. The aerosol provision device 400 comprises a housing 410 formed by a drinking vessel 450, for example a cup or mug, and a lid 411 for the vessel 450. In this example, the interior of the vessel 450 defines a first chamber 412 for containing a hot beverage, for example, tea, coffee, hot chocolate, hot squash, hot lemon juice or the like. As will be described in more detail below, in this example, heat from the hot beverage is used to heat a substance to generate an aerosol that the user can then inhale through an outlet of the lid 411. Accordingly, in this example, the device 400 has the dual function of an aerosol provisions device and a drinking vessel. It will be clear that the lid 411 and drinking vessel 450 may be separate items, and that the lid 411 could be provided separately from the drinking vessel 450.

The device 400 further comprises a second chamber 420, in the housing 410, for containing a substance, as previously described above, that is heatable to generate an aerosol and as also described above, a thermal energy transfer device 430 for transferring thermal energy from the hot liquid when the hot liquid is contained in the first chamber 412 to the substance when the substance is in the second chamber 420 to heat the substance to generate the aerosol, and an outlet 440 for the lid 411 through which aerosol generated by the substance when heated can flow.

A thermal energy transfer device 430 is arranged proximally to the second chamber 420. In this example, the energy transfer device 430 is again a heat pipe. In an example, the energy transfer device 430 may be a thermally conductive member such as a wall arranged between the first chamber 412 and the second chamber 420. The wall may be made of for example metal or metals. The energy transfer device 430 conducts heat to the chamber 420 and therefore to the substance contained within the second chamber 420. The substance contained in the second chamber 420 is heatable to generate an aerosol as described previously with respect to FIGS. 1 to 3C. In a specific example the substance contained within the second chamber 420 comprises tobacco. As explained above, the second chamber 420 may be a space defined by or at the proximal end of the energy transfer device 430. As also explained above, the substance may itself be contained in a flavor pod placed into the second chamber 420 or loose material placed into the second chamber 420.

In the example shown in FIG. 4, the lid 411 has an opening 415. In use, the first chamber 412 contains the hot liquid (i.e. a beverage) and the liquid is in fluid communication with the opening 415. The opening 415 may have a valve 417. The arrangement of the opening 415, valve 417 and vessel 450 is such that, in use, the liquid held in the first chamber 412 may pass through the valve 417.

In use, liquid contained within the vessel 450 may enter the opening 415 and contact the valve 417. The valve 417 only allows liquid to pass when a user is attempting to drink from the vessel 450. The valve 417, in an example, is a smart valve 417 which may sense for example, the location of a user's mouth over the valve 417 prior to allowing passage of liquid, or the application of pressure to the valve 417 to indicate a user's attempt to drink from the vessel 450. The smart valve 417 may sense when a user is drinking from the vessel 450 or, rather, inhaling on the outlet 440, and only directs liquid through the valve 417 when the user is drinking. The valve 417 may be mechanically or electronically operated.

Referring still to FIG. 4, the device 400 has a vent 419. The arrangement of the device 400 is such that air may pass through the vent 419 into the device 400 from outside the apparatus 400. In an example, the vent 419 may allow airflow only into the second chamber 420 which may pass over the heated substance prior to inhalation by the user. The vent 419 may be arranged to prevent or significantly hinder liquid exiting the vessel 450. The vent 419 may be a small opening, such that liquid is hindered exiting the vessel 450. Alternatively or additionally the vent 419 may have a gauze or mesh arranged within it, to help prevent liquid exiting the vessel 450 but still enabling air to enter the device 400. In an example, the vent 419 may be arranged on the vessel 450. In another example, the vent 419 may be arranged in the lid 411.

The outlet 440 may be an adjustable outlet 440 as described earlier with reference to FIGS. 3B and 3C. For concision, the description will not be repeated here. In an example, there is a mesh arranged between the outlet 440 and the second chamber 420 to prevent particulate from the substance contained in the second chamber 420 in use from being entrained in the airflow and entering the mouth of a user inhaling on the device 400. In an example, the outlet 440 may be a mouthpiece which is suitable for being received in the mouth of a user to allow the user to draw on the mouthpiece.

In the device 400, shown in FIG. 4, the vessel 450 may comprise the energy transfer device 430 and the second chamber 420. The energy transfer device 430 may be supported in the vessel 450 and the second chamber 420 may be supported by the energy transfer device 430 or the vessel 450. In an example, the energy transfer device 430 and the second chamber 420 may be supported by struts which project inwardly from the walls of the vessel 450.

In an example, the lid 411 may comprise the energy transfer device 430 and the second chamber 420. In this example, the second chamber 420 and energy transfer device may be supported by the lid 411, and be secured to the underside of the lid 411.

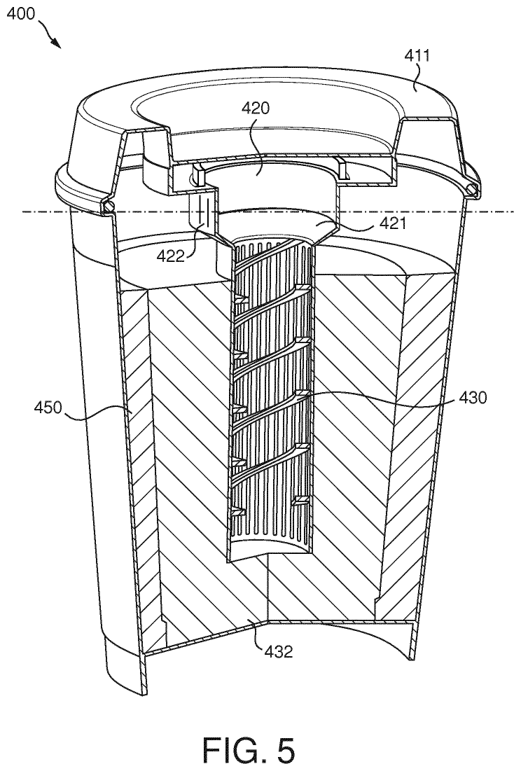

Referring now to FIG. 5, in a specific example, the device 400 comprises a drinking vessel 450 which is a standard cup, for example made of cardboard or the like, typically used as a beverage container in many commercial shops that serve hot drinks, for example, coffee shops, and the lid 411 is typically made of a plastics material, that can be releasably attached to the rim of the vessel 450.

In this example, the heat pipe 430 is releasably connectable to an underside of the lid 411 so that, in use, the heat pipe 430 is supported by the lid 411 with the end 421 of the heat pipe that defines the second chamber 420 positioned just below the lid 411.

In some examples, it is envisaged that a user may take his or her own personal lid 411 and heat pipe 430 apparatus to, for example, a coffee shop, and purchase a hot drink in a drinking cup 450 that comes with a ‘standard’ lid provided by the shop. The user may then replace the ‘standard’ shop lid with the user's own lid 411 and heat pipe 430 apparatus with a suitable substance located in the second chamber 420. The user may then use the device 400 both as an aerosol provision device and as a drinking vessel as described above.

It will be appreciated that the user may detach the heat pipe 430 from the underside of lid 411 in order to charge the second chamber 420 with substance before re-attaching the two and then attaching the lid 411 to the cup 450 with at least a lower section of the heat pipe 430 immersed in the hot beverage 432.

Different sizes of lids 411 may be available with various different diameters that correspond to the various different diameters of cups provided in typical drinking shops (e.g. small, medium and large).

In other examples, a drinking shop may provide drinks that come ready prepared in a device 400 and a user may choose to insert his or own chosen substance into the second chamber 420 so that the device 400 can then be used as an aerosol provision device as well as a drinking cup.

In some examples, the cup 450 may be of paper fiber with a low-density polyethylene plastic liner.

In the example shown in FIG. 5, the lid 411 has vapor vents 422. The vapor vents 422 are formed in the section at an end 421 of the heat pipe 430 that defines the second chamber 420. The vents 422 allow air to flow into the second chamber 420 when a user takes a draw to mix with the aerosol generated in the second chamber 420. This aerosol may then be inhaled by a user.

In other examples, the cup 450 may be a re-usable travel mug and the lid 411 a lid for such a travel mug.

In some examples, rather than being supported by the lid 411, the heat pipe 430 may be supported by, for example formed integrally with, the cup 450.

In an example, the energy transfer device 430 and the second chamber 420 may be part of a cartridge which can be removably inserted into the device 400. A user may removable secure the cartridge to any of the lid 411, the first chamber 412 or part of the housing 410 of the device 400. In an example, the second chamber 420 of the cartridge may contain, for example, tobacco but be sealed. In this case, the cartridge may be discarded after the tobacco has been used. Alternatively, the second chamber 420 may not be sealed and the, for example, tobacco can be replaced by a user after use. In this example the first chamber 412 is part of the device 400.

In an example, the cartridge may comprise both the first chamber 412 and the second chamber 420, the cartridge being removably insertable into the device 400. The cartridge in this example may also comprise the energy transfer device 430. In an example, the second chamber 420 of the cartridge may contain, for example, tobacco but be sealed. In this case, the cartridge may be discarded after the tobacco has been used. Alternatively, the second chamber 420 may not be sealed and the, for example, tobacco can be replaced by a user after use. The cartridge may be removably secured to any of the lid 411 or part of the housing 411 of the device 410.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilized and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. An aerosol provision device for generating an inhalable aerosol, the aerosol provision device comprising:
a housing containing:
a first chamber for containing a hot liquid, wherein the first chamber is fillable, by a user, with the hot liquid,
a second chamber for containing a substance that is heatable to generate the aerosol, and
a thermal energy transfer component for being immersed in the hot liquid when the hot liquid is contained in the first chamber to transfer thermal energy along the thermal energy transfer component from the hot liquid in the first chamber to the substance when the substance is contained in the second chamber in order to heat the substance to generate the aerosol; and
a mouthpiece comprising an outlet through which the aerosol may flow.

2. (canceled)

3. The aerosol provision device according to claim 1, wherein the aerosol provision device is arranged such that, when the first chamber contains the hot liquid and the aerosol provision device generates the aerosol, the housing is closed to prevent a user from accessing the first chamber.

4. The aerosol provision device according to claim 1, the first chamber comprising:
an opening to provide access to the first chamber; and
a closing mechanism for engaging with the opening,
wherein the closing mechanism is arranged to engage with the opening to prevent access to the first chamber through the opening.

5. The aerosol provision device according to claim 1, wherein the thermal energy transfer component prevents liquid held in use in the first chamber from passing into the second chamber.

6. (canceled)

7. The aerosol provision device according to claim 1, wherein the second chamber is formed integrally with the thermal energy transfer component.

8. The aerosol provision device according to claim 1, wherein the thermal energy transfer component is a heat pipe.

9. The aerosol provision device according to claim 1, wherein the second chamber is configured for containing a substance which comprises tobacco and that is heatable to generate an aerosol.

10. The aerosol provision device according to claim 1, wherein the outlet of the mouthpiece is an adjustable outlet for controlling an amount of the aerosol flowing through the outlet.

11. The aerosol provision device according to claim 1, wherein the aerosol provision device is a hot beverage vessel and the hot liquid is a hot beverage, and the first chamber is arranged for containing the hot beverage.

12. The aerosol provision device according to claim 11, comprising an opening in fluid communication with the hot beverage vessel, wherein the aerosol provision device is arranged such that in use liquid held in the hot beverage vessel may pass through the opening.

13. The aerosol provision device according to claim 12, wherein the opening has a valve, wherein the aerosol provision device is arranged such that in use liquid held in the hot beverage vessel may pass through the valve.

14. The aerosol provision device according to claim 11, the housing comprising at least one vent, wherein the aerosol provision device is arranged such that air may pass through the vent into the second chamber from outside the aerosol provision device and from the second chamber to the mouthpiece.

15. An apparatus for use with a vessel for containing a hot liquid, the apparatus comprising:
a lid for the vessel;
a chamber for containing a substance that is heatable to generate an aerosol;
a thermal energy transfer device for transferring thermal energy from the hot liquid when the hot liquid is contained in the vessel to the substance when the substance is in the chamber to heat the substance to generate the aerosol; and
an outlet for the lid through which the aerosol generated by the substance when heated can flow.

16. The apparatus according to claim 15, wherein the lid comprises an opening, wherein the apparatus is arranged such that, in use, the vessel is in fluid communication with the opening.

17. The apparatus according to claim 16, wherein the opening has a valve, wherein the apparatus is arranged such that, in use, liquid held in the vessel may pass through the valve.

18. The apparatus according to claim 15, the lid comprising at least one vent, the apparatus being arranged in use such that air may pass through the vent into the apparatus from outside the apparatus.

19. The apparatus according to claim 15, wherein the outlet is an adjustable outlet for controlling an amount of aerosol flowing through the outlet.

20. The apparatus according to claim 15, wherein the vessel is a drinking vessel and the hot liquid is a hot beverage.

21. (canceled)

22. The apparatus according to claim 15, wherein the thermal energy transfer device is a heat pipe.

23. The apparatus according to claim 15, wherein the substance that is heatable to generate an aerosol comprises tobacco.

24. (canceled)

25. (canceled)

26. A cartridge for use with an aerosol provision device for generating an inhalable aerosol; the cartridge comprising:
a first chamber for containing a substance that is heatable to generate the aerosol; and
a thermal energy transfer component which, in use, is between the first chamber and a second chamber containing a hot liquid, the thermal energy transfer component for transferring thermal energy from hot liquid in the second chamber to the substance in the first chamber.

27. The cartridge according to claim 26, wherein the cartridge comprises the second chamber.

28. The cartridge according to claim 26 wherein the thermal energy transfer device is a wall of the first chamber or is a heat pipe.

29. The cartridge according to claim 26, wherein the second chamber is part of the aerosol provision device that the cartridge is inserted into in use.

30. A system for generating an inhalable aerosol, the system comprising:
the cartridge of claim 26; and
an aerosol provision device into which the cartridge is insertable.
